(19) United States
(12) Patent Application Publication
(10) Pub. No.: US 2010/0105081 A1
(43) Pub. Date: Apr. 29, 2010

(54) DETECTING FUNGI IN HUMAN SAMPLES

(75) Inventors: Hirohito Kita, Rochester, MN (US); Diane Squillace, Rochester, MN (US); David A. Sherris, Buffalo, NY (US); Jens Ponikau, Buffalo, NY (US); Mark Swanson, Rochester, MN (US)

Correspondence Address: FISH & RICHARDSON P.C., PO BOX 1022, MINNEAPOLIS, MN, 55440-1022, US

(73) Assignee: MAYO MEDICAL VENTURES, Rochester, MN (US)

(21) Appl. No.: 11/573,527

(22) PCT Filed: Aug. 12, 2005
(86) PCT No.: PCT/US2005/028821

§ 371 Date: Dec. 28, 2009

Related U.S. Application Data
(60) Provisional application No. 60/601,427, filed on Aug. 12, 2004.

(51) Int. Cl. G01N 33/569

(52) U.S. Cl. 435/7.31

(57) ABSTRACT

Methods and materials involved in detecting fungi in human samples are provided herein.

### BACKGROUND

1. Technical Field

This document provides methods and materials involved in detecting fungi in human samples.

2. Background Information

Many different types of fungal organisms exist in nature. In addition, fungal organisms can live and grow in various types of environments.

### SUMMARY

This document describes methods and materials involved in detecting fungi in human samples. Detecting fungal organisms in a human sample can allow clinicians to confirm the presence of fungal organisms within a human to be treated. For example, the methods and materials provided herein can be used to confirm that patients suffering from chronic rhinosinusitis (CRS), asthma, or both have fungal organisms in their nasal, paranasal, sinus, and/or lung airways.

In general, this description features a method for detecting a fungal organism in a sample from a human, wherein the fungal organism produces a fungal antigen. The method includes (a) contacting the sample with an antibody to form an antibody-fungal antigen complex, wherein the antibody binds to the fungal antigen, and (b) detecting the presence or absence of the antibody-fungal antigen complex, wherein the presence of the antibody-fungal antigen complex indicates that the sample contains the fungal antigen. The fungal organism can be Alternaria alternata. The sample can be a nasal sample. The sample can be a lung sample. The sample can be a bronchial alveolar lavage sample. The human can have chronic rhinosinusitis or asthma. The fungal antigen can be an Alternaria antigen. The Alternaria antigen can be an Alt al antigen. The antibody can be immobilized to a solid support (e.g., a surface of an ELISA plate).

In another aspect, the document describes a method for detecting a fungal organism in a sample from a human, wherein the fungal organism produces fungal antigens. The method includes (a) contacting the sample with an antibody in the presence of immobilized fungal antigens under conditions wherein fungal antigens present within the sample compete with the immobilized fungal antigens for binding to the antibody, and (b) detecting the level of competition between fungal antigens within the sample and the immobilized fungal antigens by determining the level of antibody-immobilized fungal antigen complex formation. The fungal organism can be Alternaria alternata. The sample can be a nasal sample. The sample can be a lung sample. The sample can be a bronchial alveolar lavage sample. The human can have chronic rhinosinusitis or asthma. The fungal antigens can be an Alternaria antigens. The Alternaria antigens can be Alt al antigens. The immobilized fungal antigens can be Alt al polypeptides immobilized to a solid support (e.g., a surface of an ELISA plate).

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

This document provides methods and materials related to detecting the presence or absence of fungal organisms within human samples. Any type of fungal organism can be detected including, without limitation, Absidia, Aspergillus flavus, Aspergillus fumigatus, Aspergillus glaucus, Aspergillus nidulans, Aspergillus versicolor, Alternaria, Basidiobolus, Bipolaris, Candida albicans, Candida lypolytica, Candida parapsilosis, Cladosporium, Conidiobolus, Cunninahamella, Curvularia, Dreschlera, Exserohilum, Fusarium, Malbranchia, Paecilomvces, Penicillium, Pseudallescheria, Rhizopus, Schizophylum, Sporothrix, Acremonium, Arachniotus citrinus, Aurobasidioum, Beauveria, Chaetomium, Chryosporium, Epicoccum, Exophilia jeanselmei, Geotrichum, Oidiodendron, Phoma, Pithomyces, Rhinocladiella, Rhodoturula, Sagrahamala, Scolebasidium, Scopulariopsis, Ustilago, Trichoderma, and Zygomycete fungal organisms. In addition, any type of human sample can be obtained and examined for the presence of absence of fungal organisms. For example, nasal, paranasal, sinus, or lung samples can be obtained and assessed for the presence of fungal organisms.

Once obtained, a human sample is tested for the presence or absence of fungal organisms using, for example, an immunological assay. Examples of immunological assays that can be used include, without limitation, ELISAs. An ELISA can be configured such that antibodies are immobilized to a surface of the solid support to capture any fungal antigens present within a test sample. In these cases, a second antibody can be used as a detection antibody to detect the presence, absence, or level of any fungal antigens captured from the sample. Such immobilized antibodies and detection antibodies can be antibodies capable of binding to the fungal antigens within the sample. In some cases, the immobilized antibodies can be the same as the detection antibodies, with the exception that the detection antibodies are labeled.

In some embodiments, an ELISA can be configured such that fungal antigens (e.g., an Alternaria Alt al antigen) are immobilized to a surface of the solid support to compete with any fungal antigens present within a test sample for binding to anti-fungal antigen antibodies. In these cases, a second antibody can be used as a detection antibody to detect the presence, absence, or level of any anti-fungal antigen antibodies bound to the immobilized fungal antigens. Such detection antibodies can be labeled antibodies capable of binding to the anti-fungal antigen antibodies.

The invention will be further described in the following examples, which do not limit the scope of the invention described in the claims.

### EXAMPLES

### Example 1

### Detecting Fungal Organisms Within Human Nasal Secretions

Nasal secretions were collected from 9 patients with CRS and 9 normal individuals. Nasal secretions were obtained from the nasal cavities (e.g., the floor of nose, the septum, and the middle turbinate of both nostrils) using a sterile sinus secretion collector (Xomed Surgical Produces, Jacksonville, Fla.). The mean volume of secretions was 138±35 μL (mean±SEM, n=18). Each secretion specimen was extracted by adding twice the volume of 0.9% NaCl, vortexing, and centrifugation. The supernatants were stored at −20° C. An Alternaria antigen, Alt al, was measured using an ELISA kit (INDOOR Biotechnologies, Charlottesville, Va.) according to the manufacturer's procedures with a slightly modified standard curve. Total Alternaria polypeptides were measured immunochemically by inhibition as described elsewhere (Swanson et al., J. Allergy Clin. Immunol., 94:445-51 (1994)). Briefly, samples and a standard extract from Alternaria alternata competed with solid-phase Alternaria alternata polypeptides bound on ELISA plates for rabbit anti-Alternaria IgG antibodies (ALK-Abello, Round Rock, Tex.). Radiolabeled, affinity-purified, goat anti-rabbit IgG was used for detection. The results were expressed as mass protein per mL nasal secretion supernatants. The detection limits were 40 pg/mL and 12 ng/mL for Alt al and total Alternaria polypeptides, respectively.

The elevated humoral and cellular immune responses to Alternaria in CRS patients could be explained by increased airway exposure to Alternaria. All nasal secretions from 9 CRS patients contained markedly elevated levels of eosinophil MBP (4093±878 ng/mL, mean±SEM) and IL-5 (226±69 pg/mL) compared to the undetectable levels in secretions from 9 other normal individuals (p=0.0003 and 0.0092, respectively). However, both the Alternaria polypeptide, Alt al, and the total polypeptides from Alternaria did not differ between groups (243±50 vs. 208±60 pg/mL, CRS vs. normal, p=0.7076 and 231±82 vs.135±41 ng/mL, p=0.4679, CRS vs. normal, respectively).

### Example 2

### Detecting Fungal Organisms Within Human Bronchial Alveolar Lavage Samples

Bronchial alveolar lavage (BAL) samples were collected from asthmatic and non-asthmatic humans. The BAL samples were tested for the presence of fungal organisms using an ELISA kit for Alternaria Alt al antigens (Indoor Biotechnologies, Charlottesville, Va.) and culturing techniques.

The culturing and processing of the BAL specimens resulted in positive cultures in 5 of 10 samples (50%) in asthmatic patients (Table 1). In non-asthmatic BAL specimens, 9 of 18 patients resulted in positive cultures (Table 2). ELISA testing for asthmatic patients was positive in 6 of 10 (60.0%; Table 1) and 15 of 18 (83.3%; Table 2) non-asthmatic patients. A total of 14 positive fungal cultures were obtained with an average of 1.64 organisms per patient with a single sample maximum of 3 organisms. A total of 14 genera of fungi were identified. Organisms grown from asthmatic patients were the same as those grown from non-asthmatic human with the exception that Alternaria, Geotrichium sp., and Arachinoitus sp. were not identified in the tested non-asthmatic humans.

TABLE 1
Asthmatic BAL results (Alt a1 antigen and Culture).

| Patient | Sex | Age | Alt a1 | Culture Results |
|---|---|---|---|---|
| 1 | M | 87 | 0 | Alternaria |
| 2 | F | 68 | 0.017 | Negative |
| 3 | M | 31 | 0 | Penicillium sp.; Geotrichum sp.; |
| | | | | Cladosporium sp. |
| 4 | F | 53 | 0.035 | Negative |
| 5 | F | 71 | 0.016 | Penicillium |
| 6 | M | 72 | 0.001 | Paecilomyces lilacinus |
| 7 | M | 78 | 0.014 | Negative |
| 8 | F | 53 | 0 | Negative |
| 9 | M | 87 | 0 | Arachiniotus citrinus |
| 10 | F | 68 | 0.017 | Negative |

TABLE 2
Non-asthmatic BAL results (Alt a1 antigen and Culture).

| Patient | Sex | Age | Alt a1 | Culture Results |
|---|---|---|---|---|
| 09 | F | 57 | 0.011 | Cladosporium |
| 10 | M | 71 | 0.012 | Yeast |
| 11 | F | 68 | 0.017 | Aspergillus niger; Paecilomyces sp. |
| 12 | F | 64 | 0.026 | Negative |
| 13 | F | 70 | 0.026 | Aspergillus versicolor; Cladosporium; |
| | | | | Trichoderma |
| 14 | M | 58 | 0.031 | Penicillium sp.; Aspergillus niger; |
| | | | | Aspergillus Flavus |
| 15 | M | 77 | 0.04 | Negative |
| 16 | M | 27 | 0.11 | Negative |
| 17 | M | 75 | 0.15 | Candida Albicans; Aspergillus |
| | | | | fumigatus |
| 18 | F | 75 | 0.42 | Negative |
| 19 | M | 65 | 0 | Negative |
| 20 | M | 58 | 0 | Scropolariosis brumptii; |
| | | | | Acremonium sp. |
| 21 | M | 55 | 0 | Aspergillus Versicolor |
| 22 | M | 44 | 0.001 | Negative |
| 23 | F | 45 | 0.014 | Penicillium |
| 24 | F | 59 | 0.016 | Negative |
| 25 | F | 64 | 0.017 | Negative |
| 26 | M | 63 | 0.035 | Cladosporium sp. |

Overall, the presence of Alternaria was demonstrated by ELISA in 75% of patients tested. The asthmatic group had 6 of 10 (60%) samples positive, and the non-asthmatic patients scored slightly higher with 15 of 18 (83.3%) of samples being positive. Ten control ELISA samples were processed and tested. Of the controls, 0 of 10 (0%) were positive for the Alternaria antigen.

These results demonstrate that an ELISA kit can be reliably and reproducibly used to detect Alternaria fungus antigen levels not readily detected by culturing methods.

### Example 3

### Detecting Fungal Antigens

Nasal secretions were obtained under endoscopic guidance using a sterile sinus secretion collector (Xomed Surgical Produces, Jacksonville, Fla.) from CRS patients before and after treatment with either placebo or amphotericin B. Each secretion specimen was extracted by adding twice the volume of 0.9% NaCl, vortexing, and centrifugation. The supernatants were stored at −20° C. To monitor changes in fungal antigen burden with treatment, total Alternaria polypeptides in nasal secretions were immunochemically quantitated essentially as described for latex polypeptides. Briefly, samples and a standard extract from Alternaria alternata competed with solid-phase Alternaria alternata polypeptides for rabbit anti-Alternaria IgG antibodies (ALK-Abello, Round Rock, Tex.). Radiolabeled, affinity-purified, goat anti-rabbit IgG was used for detection. The results are expressed as mass polypeptide per mL nasal secretion supernatants. The detection limit of the assay was 12 ng/mL for total Alternaria polypeptides. Sufficient quantities of mucus were not available to perform all the assays for each patient. The analyses were performed based on the availability of paired samples. All nasal mucus supernatants contained Alternaria polypeptides, indicating that fungi were present (Table 3).

TABLE 3
Alternaria within human nasal secretions.

| | | Amphotericin B | |
|---|---|---|---|
| Alternaria μg/mL | Placebo | Treatment | P-value |
| Baseline | | | |
| N | 7 | 9 | |
| Median | 6.77 | 6.56 | 1.00 |
| Range | 3.91 to 10.06 | 1.33 to 25.81 | |
| After treatment | | | |
| N | 7 | 6 | |
| Median change | −0.30 | −0.30 | 0.73 |
| Range for change | −1.91 to 4.81 | −4.64 to 3.21 | |

### Example 4

### Detecting Fungal Organisms and Fungal Antigens Within Tissue Sections

Sections of formalin-fixed, paraffin-embedded tissues were deparaffinized and rehydrated. The sections were digested with trypsin (0.1%) for one hour at 37° C. The sections were washed in phosphate buffered saline and incubated overnight in normal goat serum (10%). On the following day, the sections were washed and incubated for 30 minutes at 37° C. with a 1:40 dilution of either the polyclonal rabbit anti-Alternaria antibodies or normal rabbit serum as a negative control. The sections were washed and incubated for 30 minutes at room temperature in 1% chromotrope 2R. The sections were washed again and incubated for 30 minutes at 37° C. with fluorescein-labeled goat anti-rabbit IgG antibodies (40 μg/mL). Following a final wash, the slides were mounted and coverslipped. The presence or absence of Alternaria in the tissues was analyzed using fluorescence microscopy.

Histologic slides from 12 patients were analyzed by immunofluorescence staining using anti-Alternaria antibodies as described above. Positive staining for Alternaria was observed in 6 of the 12 patients. In addition, immunofluorescence staining using anti-Alternaria antibodies demonstrated the presence of fungal elements in 12 areas of the slides, while corresponding slides stained with the Gomori methenamine silver stain did not.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for detecting a fungal organism in a sample from a mammal, wherein said fungal organism produces a fungal antigen, said method comprising:
(a) contacting said sample with an antibody in the presence of immobilized fungal antigens under conditions wherein fungal antigens present within said sample compete with said immobilized fungal antigens for binding to said antibody, and
(b) detecting the level of competition between fungal antigens within said sample and said immobilized antigens by determining the level of formation of antibody-immobilized fungal antigen complexes, wherein the presence of said competition indicates that said nasal sample contains said fungal organism.

2. The method of claim 1, wherein said fungal organism is Alternaria alternata.

3. The method of claim 1, wherein said sample is a nasal sample.

4. The method of claim 1, wherein said sample is a lung sample.

5. The method of claim 1, wherein said sample is a bronchial alveolar sample.

6. The method of claim 1, wherein said mammal is a human.

7. The method of claim 1, wherein said mammal has chronic rhinosinusitis.

8. The method of claim 1, wherein said mammal has asthma.

9. The method of claim 1, wherein said fungal antigen is an Alternaria antigen.

10. The method of claim 9, wherein said Alternaria antigen is an Alt al antigen.

11. The method of claim 1, wherein said immobilized fungal antigens are Alt al polypeptides immobilized to a solid support.

12. The method of claim 11, wherein said solid support is an enzyme-linked immunosorbent assay plate.

13. A method for detecting a fungal organism in a sample from a mammal, wherein said fungal organism produces a fungal antigen, said method comprising:
(a) contacting said sample with an antibody to form an antibody-fungal antigen complex, wherein the antibody binds to the fungal antigen, and
(b) detecting the presence or absence of the antibody-fungal antigen complex, wherein the presence of the antibody-fungal antigen complex indicates that the sample contains the fungal antigen.

14. The method of claim 13, wherein said fungal organism is Alternaria alternata.

15. The method of claim 13, wherein said sample is a nasal sample.

16. The method of claim 13, wherein said sample is a lung sample.

17. The method of claim 13, wherein said sample is a bronchial alveolar lavage sample.

18. The method of claim 13, wherein said sample is a tissue section.

19. The method of claim 13, wherein said mammal is a human.

20. The method of claim 13, wherein said mammal has chronic rhinosinusitis.

21. The method of claim 13, wherein said mammal has asthma.

22. The method of claim 13, wherein said fungal antigen is an Alternaria antigen.

23. The method of claim 22, wherein said Alternaria antigen is an Alt al antigen.

24. The method of claim 13, wherein said antibody is immobilized to a solid support.

25. The method of claim 24, wherein said solid support is an enzyme-linked immunosorbent assay plate.
